(19) United States
(12) Patent Application Publication
(10) Pub. No.: US 2002/0037593 A1
(43) Pub. Date: Mar. 28, 2002

(54) Diffraction-based cell detection using a micro-contact-printed antibody grating

(76) Inventors: Harold G. Craighead, Ithaca, NY (US); Pamela M. St. John, San Francisco, CA (US); Nathan Cady, Seattle, WA (US); Robert C. Davis, Provo, UT (US); Carl A. Batt, Groton, NY (US)

Correspondence Address: Colin D Barnitz, Jones Tullar & Cooper PC, P O Box 2266 Eads Station, Arlington, VA, 22202, US

(21) Appl. No.: 09/496,039

(22) Filed: Jan. 27, 2000

Related U.S. Application Data
(60) Provisional application No. 60/116,996, filed on Jan. 25, 1999.

(51) Int. Cl. G01N 33/543

(52) U.S. Cl. 436/518

(57) ABSTRACT

An optical biological detector is able to bind specific targeted bacterial cells by stamping an antibody grating pattern onto a silicon surface. The antibody grating alone produces insignificant optical diffraction, but upon immunocapture of the targeted cells, the optical phase change produces a diffraction pattern. Micro-contact printing provides a method for placing the antibody grating pattern directly onto a substrate surface with no additional processes or binding chemicals. Antibodies or other biologically active material may be stamped directly onto clean native oxide silicon substrates with no other chemical surface treatments. Direct binding of the antibodies to the silicon occurs in a way that still allows them to function and selectively bind antigen. The performance of the sensor was evaluated by capturing Escherichia coli O157:H7 cells on the antibody-stamped lines and measuring the intensity of the first order diffraction beam resulting from the attachment of cells. The diffraction intensity increases in proportion to the cell density bound on the surface.

### RELATED APPLICATION INFORMATION

This Application claims the benefit of U.S. Provisional Application No. 60/116,996, filed Jan. 25, 1999, the disclosure of which is incorporated herein by reference.

### BACKGROUND OF THE INVENTION

1. Field of the Invention

The present invention generally relates to a method and apparatus for producing biological sensors, and, more particularly, optical biological sensors. In one application, the sensors of the invention may be useful in detecting bacteria in food, water supplies, and the like.

2. Description of the Prior Art

Optical biological sensors have become useful in recent years in a variety of areas and provide a number of advantages over other biological sensing techniques. For example, optical biological sensors can significantly reduce the complexity of immunoassays by eliminating secondary fluorescent or enzymatic signal-generating systems. Prior art optical detection methods employ surface plasmon resonance, surface acoustic waves, and fiber optical techniques. Under the surface plasmon resonance technique, a diffraction grating is not required. However, the surface plasmon resonance process is not amenable to batch reading of samples since a baseline reading for the system must be obtained prior to the addition of analyte. The other prior art techniques also have inherent disadvantages.

One promising development in the area of optical biosensors is in the detection and measurement of diffraction patterns reflected from bioactive surfaces. The basic principle of using optical diffractive detection with a biosensor, as applied to the present invention, is illustrated in FIGS. 1a-1b. Under this principle, a laser beam 10 is directed toward a surface 12 having an antibody pattern 14 formed thereon. The antibody pattern 14 is preferably in the form of a grating or grid, having a plurality of equally-spaced, parallel lines of antibody material. The surface 12 having the antibody pattern 14 alone reflects the laser beam 10, but produces insignificant or baseline optical diffraction 15, as illustrated in FIG. 1a. However, after surface 12 has been exposed to a targeted biological substance 16, such as Escherichia coli O 157:H7, which is able to bind the antibody pattern 14, and following immunocapture of the targeted biological substance 16 in the antibody pattern 14, the reflected laser beam forms a diffracted signal 18 which is measured by a detector 20, as illustrated in FIG. 1b. A diffraction equation then may be used to determine the cell density on the antibody grating 14 based upon the degree of diffraction detected. The degree of diffraction of laser beam 10 increases in proportion to the density of the targeted substance 16 (i.e. cell density) bound on surface 12. The diffraction equation is as follows:

a(sin θ_{d}−sin θᵢ)=mλ

wherein

a is the grating period;

θ_{d} is the diffracted angle of laser beam 10;

θᵢ is the initial undiffracted angle of laser beam 10;

m is the diffraction order; and

λ is the wavelength of the laser beam 10.

A number of different types of optical biological sensors have been reported in the prior art that utilize the measurement of diffraction patterns as an assay readout. For example, the use of optical diffraction as a means to detect complex formation on a silicon wafer has been applied to the detection of choriogonadotropin in serum samples (See Tsay, Y. G., et al., Clin. Chem. 1991, v37, pp1502-1505.) In this prior study, an antichoriogonadotropin antibody was immobilized to the silicon wafer surface in a pattern, and was used to capture the antigen from sample solutions. Detection of the bound choriogonadotropin was achieved by illuminating the surface with a laser that diffracted due to the formation binding of the biological material. The sensor described by Tsay, however, required a surface that was initially entirely coated with an antibody that was then deactivated in certain areas by ultraviolet light projected through a mask so as to generate the grating pattern. Thus, reproducibility in the antibody inactivation and in the scale-up of fabrication are possible problems associated with the approach used by Tsay. Accordingly, one problem associated with the prior art which the present invention overcomes is the ability to produce discrete, regular, micron-scaled patterns of antibodies on a surface in a reliable and cost-effective manner.

In addition, a number of methods have been reported in the prior art for binding antibodies to silicon surfaces including the use of biotin, self-assembled monolayers, and chemical modification of the surface. Under the present invention, however, it has been found that antibodies can be bound to the surface of silicon in predetermined patterns without any surface modification or additional intermediary conjugates. Thus, the present invention sets forth a method and apparatus for binding antibodies and similar substances to a surface in a discrete predetermined desired pattern, and also sets forth a method and apparatus for producing an optical biological sensor which overcomes the problems and disadvantages associated with the biological sensors of the prior art.

### SUMMARY OF THE INVENTION

Under one aspect, the present invention sets forth a method and apparatus for generating a patterned, immunoreactive surface by using direct micro-contact printing. In micro-contact printing, an elastomer stamp is produced having a surface formed with printing features arranged so as to print a desired predetermined pattern. The stamp printing surface is coated with a chemical, and the chemical is transferred to a substrate in the desired pattern by pressing the stamp against the substrate. This process has specific importance to a number of emerging technologies, such as advanced tissue engineering, bio-mineralization, DNA computing, and cultured neural networks. In the performance of micro-contact printing, surfaces are chemically and molecularly patterned on a sub-micrometer scale. Micro-contact printing has been used in the past to pattern self-assembled monolayers of compounds such as hexadecanethiol and octadecyltrichlorosilane on gold and silicon-dioxide surfaces, and for chemically patterning surfaces with alkysilanes for control of cell attachment. Micro-contact printing has an advantage over conventional micron-scale patterning methods, such as photolithographic techniques, in that it requires no harsh chemicals, making it suitable for patterning biologically-active layers of substances onto surfaces.

Under another aspect of the invention, after using functional derivatizing agents as a monolayer to which antibodies were bound, it was discovered under the present invention that antibodies could be directly stamped onto a silicon surface, thereby eliminating any requirements for chemical modifications to the silicon surface. In accordance with this discovery, a silicon surface was micro-contact printed using an elastomeric stamp with a solution of antibodies in phosphate buffered saline (“PBS”). The antibodies were printed onto the surface in a grating pattern (i.e., a plurality of equally spaced, parallel lines) which consisted of a repeating pattern of 10 μm-wide lines and 30 μm-wide spaces between the lines. No additional treatments were used to block the bare silicon spaces between the antibody-stamped lines. The silicon surface acted as an attractive medium for antibodies to adhere to, and it is believed that a dipole-dipole (van der Waals) interaction exists between the antibodies and the hydroxyl-terminated surface. In addition, it has been found that antibodies bound to a silicon surface remain able to recognize and bind their antigen efficiently and specifically. Thus, under the present invention, micro-contact printing of the antibodies onto the silicon surfaces, the surfaces were exposed to a solution containing target cells. After the target cells were given time to bind to the antibodies, the surfaces were washed with saline solution, and the resulting complexes remained bound to the silicon surfaces within the bounds of the printed pattern. Optical diffraction was then used to determine the degree to which the target cells had bound to the antibody grating.

Consequently, the present invention provides a method and apparatus for generating a micron-scale (i.e., individual features on a scale of less than 100 μm) diffraction grating made strictly from biological molecules. The diffraction pattern is established because the target antigen binds only to the antibody-stamped region. The diffraction observed when a laser is directed at the micron-scale grating is believed to be due to the phase difference between adjacent regions on the surface of the substrate. Micro-contact printing is a simple means to generate micron-scale patterns on surfaces, and typically exploits the use of self-assembling monolayers to create a reactive surface. Recently, in the prior art, more elaborate methods of directing antibodies to surfaces have been achieved using microfluidics. However, under the present invention antibodies can be directly printed onto a silicon surface, and they are believed to be bound through dipole interactions. This simple means to generate an immunoreactive microstructured surface eliminates any complex prior treatment of the surface and any complications due to potential interactions with a functionalized surface. In addition, the optical diffraction measurements are not susceptible to small defects. Therefore, any non-uniformities resulting from the stamping process are not critical to the measurements. Although the present invention cannot positively exclude any desorption of the antibodies from the surface, the interaction between the micro-contact-printed antibodies and the silicon surface has been found to be sufficiently robust to survive the subsequent process and yield a consistent, detectable signal.

Accordingly, under an additional aspect, the present invention sets forth a simple sensor which may have value in the detection of pathogenic bacteria, including E. coli O157:H7, as well as any analyte whose mass is sufficient to generate a diffraction signal when bound on a silicon surface. This sensor may have value in detecting potentially deadly bacteria in food, water supplies, or the like. The sensor is able to capture and detect minute quantities of bacteria, and, by using a laser and detector device, may be used to monitor the safety of food processing facilities, restaurants, or other locations where bacterial contamination is a concern. The sensor may be constructed having an array comprised of a plurality of individual grids of different antibodies for enabling the sensing of a plurality of different types of bacteria by a single sensor. In addition, the basic principles of the sensor, as set forth herein, may be applied to a number of other areas, such as DNA recognition, medical research, or the like.

### BRIEF DESCRIPTION OF THE DRAWINGS

The foregoing and additional objects, features, and advantages of the present invention will become apparent to those of skill in the art from a consideration of the following detailed description of preferred embodiments of the invention, taken in conjunction with the accompanying drawings.

FIGS. 1a-b illustrate the principle of using a diffraction pattern to detect cells attached to an antibody grating, and show how the sensor of the invention may be implemented. Under the invention, the diffraction pattern was observed by illuminating the grating about 10 degrees off normal with a 632.8 nm wavelength He:Ne Laser.

FIGS. 2a-2c illustrate the method of producing the sensor of the invention. A silicon grating master (not shown) was generated using contact photolithography. This master was used to create an elastomer stamp, as illustrated in FIG. 2a. The stamp was coated with antibodies, as illustrated in FIG. 2b. The coated stamp was then pressed against the surface of a silicon substrate for a period of time. The stamp was then removed from the surface, leaving antibodies on the surface in a pattern of a plurality of parallel 10 μm-wide antibody stripes separated by 30 μm-wide spaces, as illustrated in FIG. 2c.

FIG. 3 illustrates a fluorescence photomicrograph of anti-E. coli0157:H7 antibody-stamped silicon surface. The silicon surface was stamped with 10 μm-wide lines of anti-E. coli0157:H7, and, to enable visualization of the stamping, anti-goat antibodies conjugated to fluorescein were introduced and allowed to bind with the stamped antibodies. After washing the surface was photographed at 100× magnification.

FIG. 4 illustrates an Atomic Force Microscopic (“AFM”) image of an anti-E. Coli 0157:H7 antibody-stamped silicon surface. AFM was operated in the tapping mode and the height scale posted on the right side of the image.

FIG. 5 illustrates a photomicrograph of E. coli 0157:H7 bound to an antibody-stamped silicon surface. The silicon surface was stamped with 10 μm-wide lines of anti-E. coli0157:H7 antibodies. E. coli0157:H7 were then allowed to bind for 30 minutes with the antibodies, and the surface was then washed with saline solution. The E. coli cells were stained with acridine orange and photographed at 200× magnification.

FIG. 6 illustrates the diffraction intensity from increasing E. coli0157:H7 cells bound to the silicon surface stamped with anti-E. coli0157:H7 antibodies. The silicon surface was stamped with 0.1 mg/ml anti-E. coli0157:H7 antibodies and then from 104 to 107 CFU of E. coli0157:H7 allowed to bind. The surface was illuminated with 632 nm laser and the 1° diffraction measured. The cell number on the surface was plotted as a function of diffraction intensity.

FIG. 7 illustrates an additional embodiment of the invention having an array of a plurality of stamped grating pattern areas, with each grating area being formed by stamping different biologically active materials, such as antibodies, onto the surface. Each different grating area is able to bind a different targeted biological substance, such as a different type of bacteria.

### DETAILED DESCRIPTION

The present invention sets forth an optical biological sensor and a method and apparatus for producing the biological sensor. The biological sensor is useful in the detection of bacteria or other targeted biological substances. The sensor comprises a substrate surface which, using micro-contact-printing, is patterned with a bioactive material, such as an antibody, in a manner which does not create substantial diffraction when the surface is illuminated with a laser beam at a predetermined angle. The bioactive material coated onto the surface is chosen so as to be able to bind with a targeted biological substance of interest, such as a bacteria. When the bioactive material is exposed to the targeted substance, the targeted substance binds with the bioactive material, and, by its presence on the substrate, produces a detectable change in the intensity of the diffraction of the laser beam, thus indicating the presence of the targeted substance. For a given diffraction order, the greater the intensity of the diffracted laser beam, the greater the density of the targeted substance bound on the surface.

### EXAMPLE

Production of Master for Casting the Stamp

A silicon master (not shown) was generated using contact photolithography under a method described in more detail in co-pending U.S. patent application Ser. No. 1BA, filed Jan. 6, 2000, and entitled “PATTERNED PROTEIN LAYERS ON SOLID SUBSTRATES BY THIN-STAMP MICRO-CONTACT PRINTING”, Atty. Docket No. CRFD2254, and described in U.S. Provisional Application No. 60/115,136, filed Jan. 7, 1999, having the same title, both assigned in their entirety to the same assignee as herein, and the disclosures of which are incorporated herein by reference. The master used in the Example was configured to enable the production of a cast elastomer stamp for printing a plurality of equally-spaced, parallel 10-μm wide lines with 30-μm wide spaces in a grating-like pattern. The period of this grating (i.e., the distance between adjacent lines) was not optimized for the wavelength of the laser used in the Example. However, such optimization of the period is contemplated under the invention. For instance, a stamped pattern having a plurality of 5-μm-wide lines separated by 5-μm-wide spaces was found to have a superior signal, while improving the signal-to-noise ratio, when used with the laser of the Example.

In brief, to produce the master in the Example, a silicon (100) wafer (not shown) was used having a native oxide coating of approximately 15-20A. The master was generated using Shipley S1813 photoresist (2μm thick) spun onto the silicon wafer. Grooves and ridges were formed in the master using contact photolithography through a mask having a plurality of 10-μm-wide lines separated by 30-μm-wide spaces. After exposure to 405-nm light, the wafer was developed for 1 minute in Shipley MF312 diluted 1:1 in water, and then exposed to a fluorinated trichlorosilane vapor for 30 minutes to passivate the exposed 10-μm-wide grooves formed in the silicon wafer surface.

Production of the Stamp

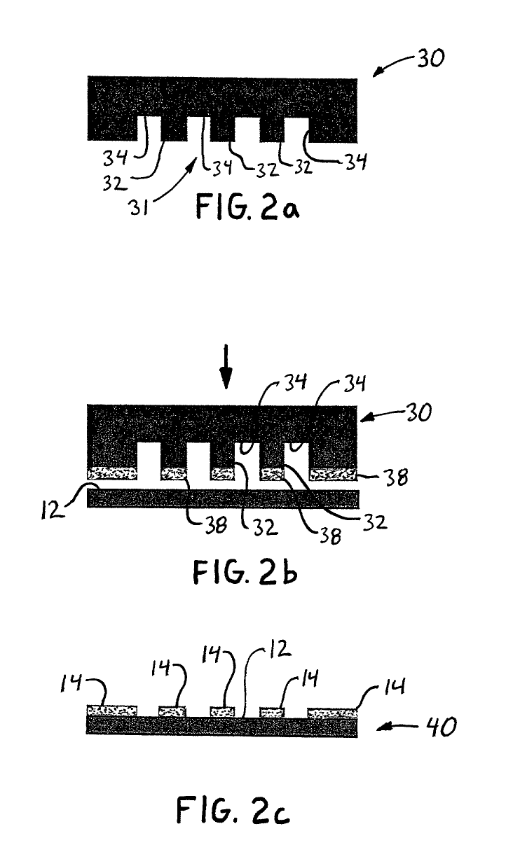

Following production of the master, a micro-contact stamp was produced by casting using Sylgard 184 silicone elastomer (Dow Corning) (poly(dimethylsiloxane)) mixed with a curing agent. The micro-contact stamp used in the present invention may be simply a pure elastomer casting of the master, or, advantageously, the present invention may utilize a rigid support as a backing for the stamp and may have only a thin elastomer layer, as set forth in the above-referenced-and-incorporated-herein co-pending applications. The thin stamp design eliminates possible sagging of the stamp surface between printing features, and, thus, eliminates any problem of printing in undesired areas. In addition, the invention is not limited to the particular elastomer for producing the stamp, and other suitable materials may be used as the stamp of the invention. As illustrated in FIG. 2a , the stamp 30 produced in the Example included printing features 31 formed as a plurality of elongate raised parallel ridges 32 which were 10-μm wide and which were separated by 30-μm-wide spaces 34 to enable the printing of parallel 10-μm wide lines spaced 30-μm apart in a grating or grid-like pattern.

Pretreatment of the Silicon Substrate

A single-crystal silicon wafer was chosen as the substrate for the Example, although other substrates, such as glass, silicon oxide, and other ceramics, polymers, metals, and the like will be apparent to those skilled in the art, so long as the antibodies, or other biological materials of interest, are able to bind to the substrate surface by micro-contact printing, and so long as the material of the substrate does not disrupt the measurement of change in diffraction in the finished sensor. In particular, other oxide surfaces may be used as the substrate of the invention since it is believed that the antibodies probably interact with the native oxide on the silicon surface, rather than the silicon. Thus, silicon having a native oxide coating is preferred as the substrate because it is an attractive medium for antibodies to adhere to; it may be produced having an extremely flat surface, and therefore is ideal for optical measurements; and it is believed that a dipole-dipole (van der Waals) interaction exists between the antibodies and the hydroxyl-terminated surface. In addition, it has been found under the present invention that antibodies bound to a silicon surface remain able to recognize and bind their antigen efficiently and specifically. In the present Example, the native oxide silicon surface 12 was treated with methylene chloride, ethanol, and finally distilled water before being stamped, as described below.

Micro-Contact Printing

Immediately before beginning the micro-contact printing procedure, a temporary increase in hydrophilicity and protein adsorption of the stamp is achieved by treatment in a low-temperature plasma cleaner/sterilizer (Harrick Scientific, P.C.-32G) evacuated with a mechanical roughing pump, as described in the above-referenced co-pending applications. The rf level was set to high, and the plasma was left on for 30 seconds.

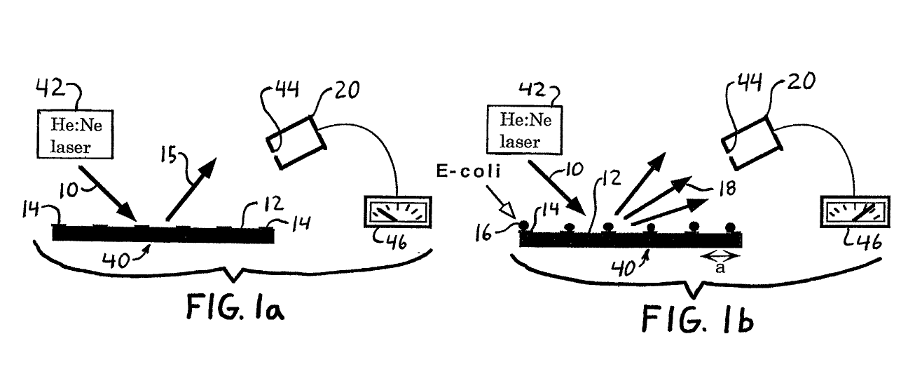

Following hydrophilization of the printing features of stamp 30, the antibody solution [0.001-0.1 mg/ml anti-E. coli0157:H7 antibodies (KPL, Gaithersburg, Md.) in a phosphate-buffered saline solution, pH 6.5] was swabbed or pipetted onto the stamp printing surface, ensuring that the printing surface of the stamp was fully wetted by the liquid. Excess liquid was removed with a pipet and finally the stamp was dried with a stream of nitrogen gas at 40 psi, leaving a layer 38 of antibodies on the printing surface of stamp 30, as illustrated in FIG. 2b. Stamp 30 was then brought into contact with silicon substrate surface 12 and held there under pressure for 30 minutes to give antibody layer 38 time to bind with silicon surface 12 . Stamp 30 was then removed leaving an antibody layer 14 adhered to silicon surface 12 in a pattern corresponding to the pattern of the printing features 31 (actually a mirror-image of the pattern of the printing features 31) of stamp 30 , thereby creating a sensor 40, as illustrated in FIGS. 1a-b and 2c. For testing purposes in the Example, a plurality of such sensors 40 were produced in the manner described above.

Surface Characterization

To facilitate visualization of the antibody pattern, and to verify that the goat anti-E. coli O157:H7 antibodies were bound only to the stamped areas, anti-goat secondary antibodies conjugated with fluorescein were allowed to bind to the surfaces of the antibodies 14 on the surface of sensor 40. The antibodies 14 were localized using anti-goat fluorescein-labeled antibodies (U.S. Biochemical, Cleveland, Ohio) diluted 1:500. The anti-goat antibodies were allowed to incubate for 1 hour and, after washing with PBS with 1% Tween, observed with a with a Nikon Labophot-2 light microscope fitted with an episcopic-fluorescence attachment EFD-3 (Nikon Corp., Tokyo, Japan).

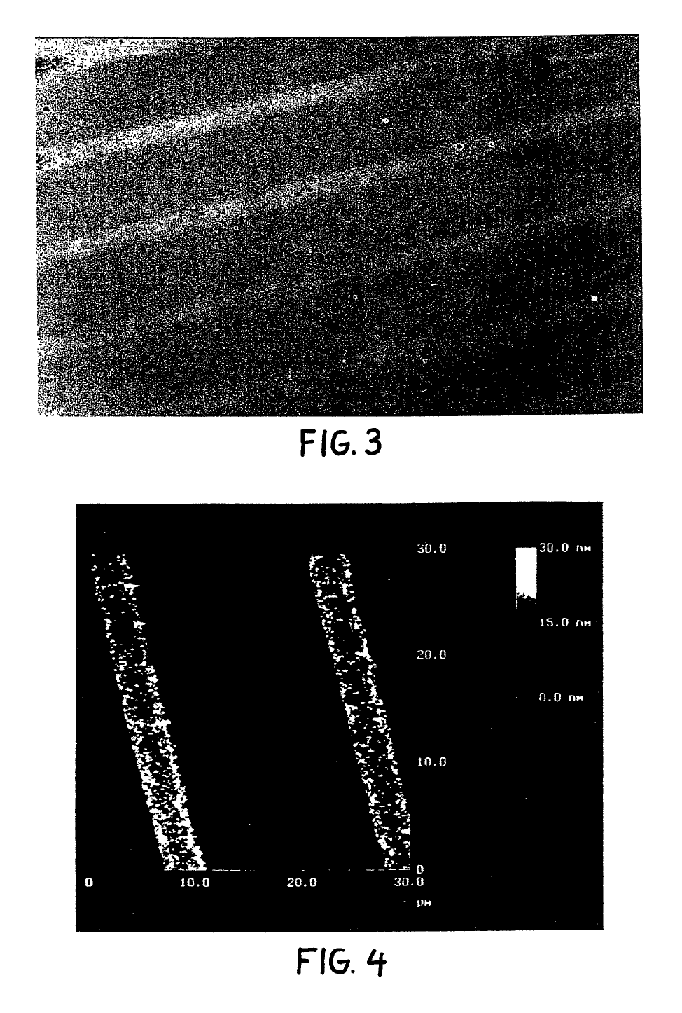

FIG. 3 shows a fluorescence micrograph of the surface 12 of sensor 40 treated with the anti-goat secondary antibodies. The secondary antibodies bound specifically and only in the stamped anti-E. coli O157:H7 regions of the surface of sensor 40. Nonspecific binding of these antibodies to the silicon surface was reduced by the inclusion of Tween into the buffer. Intense regions of fluorescence were observed, and the definition of the stamped lines at this resolution verifies that it is possible to stamp biological molecules directly onto a silicon surface using the micro-contact printing process. Variations in the fluorescence intensity over the entire sample area are a result of an incomplete coverage.

Further characterization of the antibody-stamped surface 12 was obtained using atomic force microscopy (“AFM”), as illustrated in FIG. 4. The AFM was operated in the tapping mode and the height scale is posted on the right side of the image of FIG. 4. The height of the antibody-stamped regions observed ranged from 0 to 10 nm, and the regions were not uniform in coverage. The maximum height of the stamped surface was found to be consistent with the dimensions of immunoglobulin molecules (i.e., IgG, ˜100 A), suggesting a single, albeit incomplete, monolayer was deposited. Holes in the antibody-stamped areas 14 are evident, and the edge boundary varies. However, despite these localized variations, the sample sensors 40 were able to effectively bind E. coli O157:H7 in quantities sufficient to measure by diffraction, as described below.

Exposure of Sensors to E-coli

Escherichia coli O157:H7 was cultured in Luria broth and the number of colony-forming units determined by plating onto Luria agar. The cells were diluted in PBS (pH 7.4) with 1% Tween, and a sufficient volume of the solution (˜1 ml) was pipetted onto sensor 40 to cover the entire surface. The cells were left on the surface for 10-15 minutes and then rinsed in PBS. For direct microscopic visualization, cells were stained with acridine orange (1 mg/ml) for 10 minutes, and, after washing, observed microscopically at 200× magnification. After each step, the surface was examined under an optical microscope to check for uniformity.

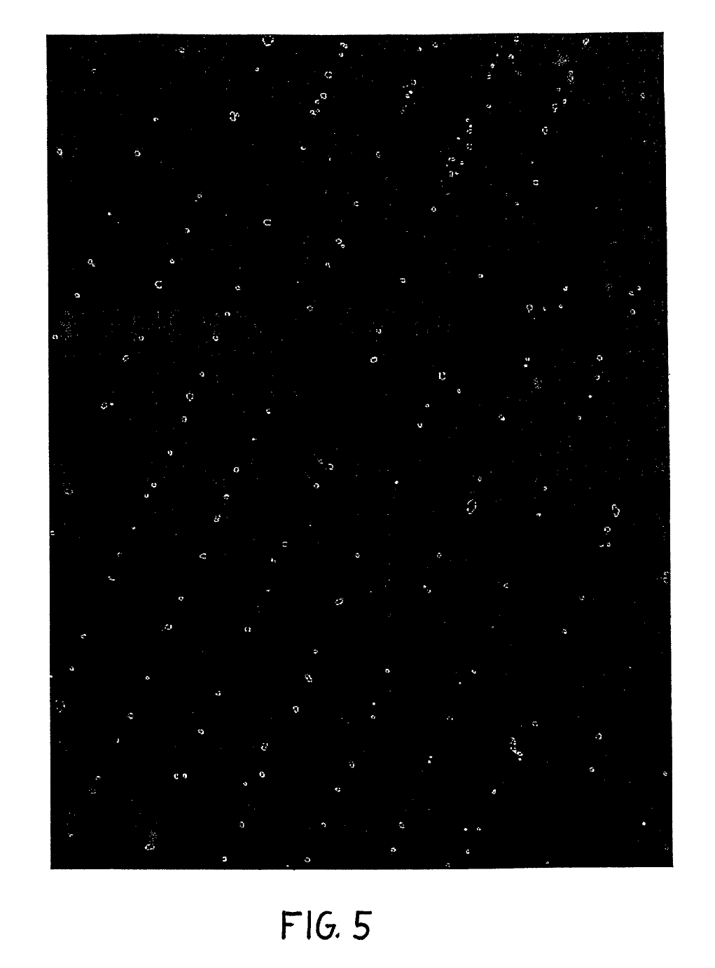

Fluorescence microscopy was used to directly observe acridine orange nucleic acid-stained cells attached to the antibodies. FIG. 5 is a photomicrograph illustrating E-coli cells bound to the surface of an antibody-stamped sensor 40. The cells align in rows corresponding to the grating spacing. E. coli O157:H7 bound only to regions of the silicon surface 12 that had been stamped with antibodies 14 . The antibodies 14 stamped on the sensor 40 were shown to effectively and rapidly capture E. Coli O157:H7 from solution with incubation times of less than 30 minutes.

Diffraction Measurements

The silicon substrate 12 was held in a clip mounted on a x, y, z and θ stage (not shown). As illustrated in FIGS. 1a-b, diffraction measurements were performed using a 632.8-nm He—Ne laser 42 focused to 1-mm diameter on a masked area of antibody pattern 14 . Masked areas were ˜1.3 mm in diameter, allowing measurement of constant areas of the sample. As illustrated in FIG. 1b, once the E. coli-bound surface 12 of sensor 40 was illuminated with laser beam 10, an aperture 44 was placed in the path of the diffracted light beam 18 so as to collect only the first-order signal (m=1). A silicon detector 20 was placed directly behind the aperture 44 so that the first-order signal was centered on the 5 mm×5 mm active area. The detector 20 was connected to a power meter 46 (Newport digital power meter model 815), and the intensity of the signals were measured in microwatts. As a baseline control, measurements of the intensity from the specular (m=0) order were taken using a neutral density filter (not shown) with an attenuation of 55 times the unfiltered beam. After diffraction measurements were taken, the number of cells within the masked regions was counted to determine the bound cell concentration per unit area.

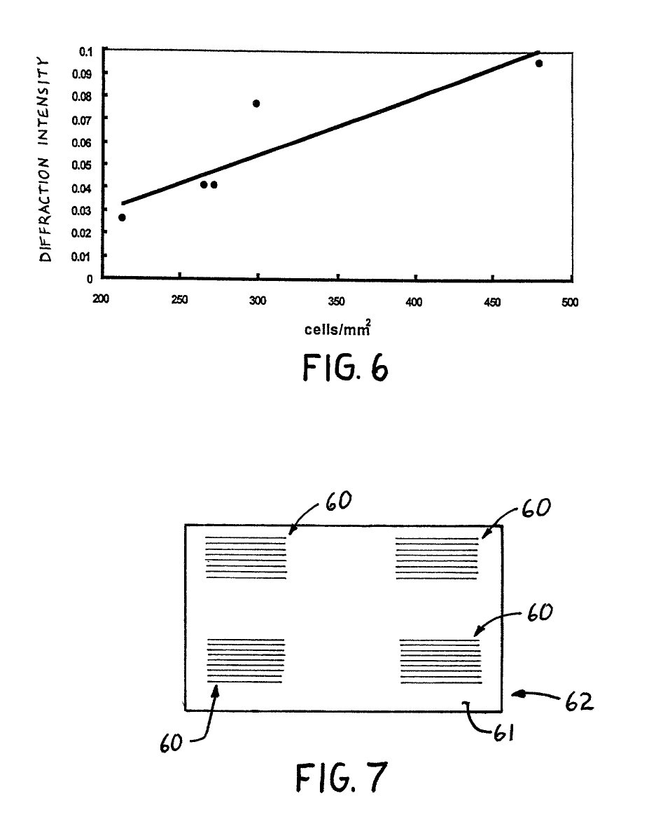

A direct measure of the bound E. coli O157:H7 cells on the antibody grating was obtained by measuring the diffraction intensity. Power meter 46 was used to measure the first-order diffraction of a He—Ne laser beam from an antibody-stamped sample at different cell coverages. The diffraction pattern present with cells attached to the antibody grating could easily be seen by eye. A linear relationship between the diffraction intensity and the number of bound cells is illustrated in the graph of FIG. 6. The diffraction increased from 0.02 to 0.097 over a range of 210-470 cells/mm². This graph was generated by taking the diffraction measurements of antibody-stamped samples to obtain a background reading for each cell concentration. As a control sample, when an equivalent number of Salmonella were incubated with the antibody-stamped sensor 40, no bound cells or any diffraction signal over background was observed (data not shown).

Discussion of Results and Applications of the Invention

Direct micro-contact printing is a simple means to generate a patterned, immunoreactive surface suitable for a variety to applications. An analyte bound to the patterned surface will generate a diffraction pattern if it has sufficient scattering cross section, as illustrated in FIG. 1b. An elastomeric stamp was used to micro-contact print a solution of antibodies in PBS onto a silicon surface in a grating or grid-like pattern. The grating consisted of a repeating pattern of 10-μm lines and 30-μm spaces, and no additional treatments were used to block the bare silicon spaces between the antibody-stamped lines or to bind the biological material to the surface 12 . The period of this grating was not optimized for the wavelength used. The silicon surface is an attractive medium for antibodies to adhere and it is believed that a dipole-dipole (van der Waals) interaction exists between the antibodies and the hydroxyl-terminated surface. Antibodies bound to the surface remained able to recognize and bind their antigen efficiently and specifically. The printed surfaces may be washed in a saline solution after the target cells are allowed to bind to the antibodies, and the complexes will remain bound to the substrate surface.

Thus, under the present invention, a diffraction grating made strictly from biological molecules was generated. The diffraction pattern is established because antigen is bound only to the antibody-stamped region. It is believed that the diffraction observed when a laser is used to illuminate the micrometer-scale grating is most likely due to the phase difference between adjacent regions on the surface. The micro-contact printing method and apparatus advanced by the present invention is a simple means to generate micrometer-scale patterns on surfaces and typically exploits the use of self-assembling monolayers to create a reactive surface. Under a further aspect of the invention, antibodies can be directly printed onto the surface of silicon and they are probably bound through dipole interactions Given the relatively nonspecific nature of this interaction it is not unexpected that there will be variability within the context of antibody coverage and functionality of the bound antibodies. Variability in the interactions between silicon and proteins has been reported, as well as means to improve protein adherence to the surface using chemical modification with aminosilane. Thus, opportunities to improve the micro-contact printing technique to generate optical biosensors, will be apparent to those skilled in the area of the invention, and may include the use of modified surfaces, additional chemicals, or the like, for improving the adherence of the biological materials to the particular substrates used.

From the foregoing, it will be apparent that, micro-contact printing is a simple means to generate an immunoreactive microstructured surface that eliminates any prior treatment and complications due to potential interactions with a functionalized surface. A predetermined bioactive pattern may be printed in a micron scale. The optical diffraction measurements used in the present invention are not susceptible to small defects, and, therefore, any nonuniformities resulting from the stamping process are not critical to the measurements. Although it is possible that some of the antibodies may desorb from the surface, the interaction between the micro-contact printed antibodies and the silicon surface is sufficiently robust to survive the subsequent process and yield a consistent, detectable signal. In summary, the simple sensor set forth herein may have value in the detection of pathogenic bacteria including E. coli O157:H7 as well as any analyte whose mass is sufficient to generate a diffraction signal when bound on a silicon surface. Thus, it will be apparent that any number of biologically-active materials may be substituted for the antibodies of the Example for attracting or binding any number of particular targeted biological substances. For example, patterns of nucleotide sequences may be stamped onto a surface using micro-contact printing. The surface may then be exposed to a solution containing DNA and diffraction detection used to determine whether complimentary DNA has bound to the particular nucleotide sequence. Other uses for the detector of the invention will also be apparent to those skilled in the area of the invention.

Additional Embodiments of the Invention

Under additional embodiments of the invention, a plurality of different antibodies may be printed in an array of plural grating pattern areas 60 on a single substrate surface 61, as illustrated in FIG. 7. Under this embodiment, a single sensor 62 may include an array of any number of different grating areas 60, with each grating area 60 being a stamping of a different biologically-active material, such as different antibodies. Thus, each area 60 is capable of detecting a different particular targeted biological substance, such as a different bacteria. In this manner, a single sensor 62 may be produced for detecting multiple targeted substances, such as multiple types of bacteria. Sensor 62 may then be placed in a strategic location, such as in a food processing facility. This sensor 62 may be periodically monitored by a computer controller (not shown) by periodically scanning the sensor with a laser beam. If a change in diffraction is detected, the computer controller may display an alarm, or other appropriate action may be taken.

Under an additional embodiment of the invention, the periods for the grating areas may be varied or tuned to specific wavelengths. Certain grating periods are more sensitive to certain laser wave lengths. For example, a period of 5 μm-wide lines spaced 5 μm apart created a stronger signal than 10 μm-wide lines spaced 30 μm apart in the Example set forth above. Thus altering or optimizing the grating period for the wavelength of the laser used is desirable for maximizing the diffraction signal and improving signal-to-noise ratio. It is contemplated that the width of the lines used to form the gratings of the invention may be as small as 1 μm or even smaller, and as large as 100 μm or anywhere in between, with the spaces between the lines being of a like range of dimensions.

Accordingly, while the foregoing disclosure sets forth exemplary embodiments of the present invention, it is to be understood that the invention is not limited to the particulars of the foregoing embodiments, but is limited in scope only as set forth in the following claims.

What is claimed:

1. A method for producing an optical biological sensor, said method comprising:
providing a stamp having printing features projecting therefrom;
providing a substrate having a surface;
adhering a bioactive material to said printing features;
pressing said printing features against said surface of said substrate to transfer said bioactive material to said surface in the locations of said printing features thereby forming the sensor.

2. The method of claim 1 wherein said printing features form a pattern, and further including the step of transferring said bioactive material to said substrate in a mirror-image pattern corresponding to the pattern of said printing features.

3. The method of claim 1 further including the step of producing a grid-like pattern of said bioactive material when pressing said printing features against said surface of said substrate, said grid-like pattern comprising a plurality of generally parallel lines of said bioactive material.

4. The method of claim 1 wherein said elastomer is poly(dimethylsiloxane), and further including the step of rendering said elastomer more hydrophilic prior to adhering the bioactive material to said printing features.

5. The method of claim 1 further including the step of providing a bioactive material which is capable a binding with a particular target substance.

6. The method of claim 5 further including the step of providing a laser and a diffraction detector in the vicinity of said substrate for illuminating said surface of said substrate with said laser to detect said target substance.

7. A sensor for use in detecting a targeted biological substance, said sensor comprising:
a substrate, said substrate having a surface; and
a biologically active material printed on said surface of said substrate in a predetermined pattern, wherein said biologically active material is able to bind the targeted substance thereto, but the targeted substance does not otherwise substantially bind to said substrate.

8. The sensor of claim 7 wherein said substrate is silicon having a native oxide surface.

9. The sensor of claim 7 wherein said biologically active material includes antibodies capable of binding the targeted biological substance.

10. The sensor of claim 7 wherein said predetermined pattern comprises a gridlike pattern including of a plurality of parallel lines of said biologically active material.

11. The sensor of claim 10 wherein said plurality of lines are between approximately 1 μm and 100 μm in width and spaced approximately 1 μm to 100 μm apart.

12. The sensor of claim 7 wherein the targeted substance is a specific bacteria and said biologically active material is a substance capable of binding the specific bacteria.

13. The sensor of claim 7 wherein the relationship between said biologically active material and said target substance is such that if said biologically active material is exposed to said target substance and said biologically active material is illuminated by a laser beam, the degree of diffraction of said laser beam changes from a first value prior to exposure to the target substance to a second value following exposure to the target substance.

14. A method of detecting bacteria, said method comprising:
providing a stamp having a printing surface, said printing surface having printing features formed in a pattern;
adhering antibodies to the printing features of said stamp;
providing a substrate having a surface;
transferring said antibodies to said surface by pressing said printing features against said surface, so that said antibodies adhere to said surface in a configuration corresponding to said pattern;
exposing said antibodies to bacteria such that the bacteria binds to said antibodies; and
optically detecting the presence of said bacteria.

15. The method of claim 14 wherein said step of optically detecting the presence of said bacteria includes illuminating the surface of said substrate with a laser beam.

16. The method of claim 15 further including the step of detecting a change in diffraction of said laser beam if said bacteria are present.

17. The method of claim 16 further including the step of providing a detector and a power meter for detecting a change in diffraction of said laser beam.

18. The method of claim 14 further including the step of providing a stamp having printing features, said printing features including in a grid-like pattern for producing a plurality of generally parallel lines of said antibodies on said substrate.

19. The method of claim 14 wherein a plurality of stampings are performed on a single substrate surface adjacent to one another, and wherein each stamping is comprised of different antibodies for binding different types of bacteria.

20. The method of claim 14 further including the step of providing a stamp having printing features, said printing features including in a grid-like pattern for producing a plurality of generally parallel lines of said antibodies on said substrate, said parallel lines being between approximately 1 μm and 100 μm in width and spaced approximately 1 μm to 100 μm apart.
